(19) United States
(12) Patent Application Publication
(10) Pub. No.: US 2010/0291041 A1
(43) Pub. Date: Nov. 18, 2010

(54) BACTERIOPHAGE PREPARATION AND USE

(75) Inventors: Neithard Dahlen, Butzbach (DE); Gabriele Ruppert-Seipp, Marburg an der Lahn (DE)

Correspondence Address: SENNIGER POWERS LLP, 100 NORTH BROADWAY, 17TH FLOOR, ST LOUIS, MO, 63102, US

(73) Assignee: INNOVATIONS-TRANSFER UPHOFF GMBH & CO. KG, Marburg an der Lahn (DE)

(21) Appl. No.: 12/863,459

(22) PCT Filed: Jan. 16, 2009
(86) PCT No.: PCT/EP09/00249

§ 371 Date: Jul. 29, 2010

(30) Foreign Application Priority Data
Jan. 18, 2008 (DE) 10 2008 005 193.4

(51) Int. Cl. A61K 35/76; A61L 2/00

(52) U.S. Cl. 424/93.6; 422/20

(57) ABSTRACT

The invention relates to a bacteriophage preparation having at least one bacteriophage in alkaline buffer solution, said bacteriophage being specifically effective against at least one bacterial strain, in combination with at least one surface-active agent that is preferably a polyhexamethyl biguanide (polyhexanide) stabilized in an alkaline range between pH 7.5 and pH 9.0. The invention further relates to the use of such a bacteriophage preparation for the production of a pharmaceutical, particularly for therapeutic, preventative, and disinfecting antibacterial use. Finally, the invention also relates to a disinfection method using the bacteriophage preparation together with low-frequency ultrasound.

The invention relates to a bacteriophage preparation according to the preamble of claim 1 and various uses thereof.

European Pat. Appl. No. EP 0 414 304 B1 describes a bacteriophage preparation from an aqueous composition of at least 100 particles/mL of a bacteriophage capable of lysing one or more types of bacteria, a nonionic surfactant, and a neutral salt. The bacteriophages possess the well-known action of lysing certain antibiotic-resistant bacteria. The surfactant as a surface-active substance is used to improve the wetting of the surface to be treated and to solubilize and remove dirt. The neutral salt, primarily 0.01 to 2.0% by weight sodium chloride, is said to improve the storage stability. Said composition is to be used as toothpaste, mouthwash, household cleaner against household bacteria (e.g., in toilet bowls), and as a skin care product against pathogenic skin bacteria. Phage-compatible fragrances, flavors, solvents, dyes, preservatives, bactericides, adsorbents, fillers, and other additives that are commonly used for a specific application are used as possible additional additives.

Furthermore, it is disclosed in EP Pat. No. EP 0 700 249 B1 and Unexamined German Pat. Appl. No. DE 100 12 026 that polyhexamethylene biguanide (polyhexanide) as a cationic surfactant has a high, antiseptically usable activity with a simultaneously low toxicity. On this basis, a plurality of applications with this active substance were developed on the market, but it was pointed out even in EP 0 700 249 B1 that polyhexanide has an activity against certain bacteria (especially Pseudomonas aeruginosa) that is lower by a factor of nearly 10, in comparison with, e.g., Staphylococcus aureus.

It has also been described in detail with use of examples even in EP 0 700 249 B1 that, e.g., during the use of polyhexanide in wounds a considerable loss of action can occur, which is caused by the albumin present there; our own microbiological tests on the effect of albumin confirm a loss of action by the factor of about 10. In contrast to polyhexanide, current studies [Vautz D. et al.: Lytic effectiveness of MRSA-specific phages against the 41 defined national reference strains and 143 clinical MRSA isolates. ZfW (2007) 5:280-287], show that in the case of bacteriophages albumin (bovine serum albumin, BSA) even produced an increase in the lytic activity by >1 to >3 log steps at comparable E/T ratios and starting bacterial concentrations.

The gram-negative Pseudomonas species, particularly Pseudomonas aeruginosa, is the cause of infections in humans and animals. Staphylococci as gram-positive bacteria colonize humans depending on the condition of their skin up to 20% in the case of healthy skin and up to 100% in the case of predamaged skin such as, e.g., atopic dermatitis or particularly the presence of wounds. Further, staphylococci are among the most frequent causative agents of nosocomial infections.

Hospital-acquired infections (nosocomial infections) constitute a major part of all complications occurring in hospitals and therefore have a significant effect on the quality of medical and nursing care for the patients. The most frequent types of nosocomial infections in the intensive care unit are ventilator-associated pneumonia, intra-abdominal infections following trauma or after surgical interventions, and bacteremias caused by intravascular foreign bodies.

The occurrence of strains with resistance to commonly employed antibiotics therefore has a particular shattering effect in connection with pneumonia and sepsis and the infection of implants and of wounds with this bacterium.

Methicillin-resistant Staphylococcus aureus (MRSA) is the most important causative agent of nosocomial infections, whose incidence since the first occurrence in 1963 has increased worldwide and whose strains have become widespread in Europe, some strains worldwide as well. In Germany, there was an increase from 1995 to 2001 from ˜8% to ˜20% of the proportion of MRSA of all S. aureus isolates. In this regard, the MRSA incidence in Germany depending on the survey (EARSS; PEG; GENARS; SARI; KISS) and particularly in different hospitals and within a hospital depending on the risk area varies from 0 to 35%, in individual cases up to 60%. Intensive care/surgical wards are most prominent in this regard and the higher mortality due to MRSA infections has been substantiated [Melzer M et al., Clin Infect Dis 2003; 37:1453-1460]. Further, outside hospitals, so-called community-acquired MRSA (cMRSA) occur, which are associated with pathogenicity properties such as necrotizing skin/soft tissue infections and necrotizing pneumonia and differ basically from the epidemic nosocomial MRSA in genotype. Clonal MRSA lines with resistance to macrolides and lincosamidines, gentamicin, and in part to oxytetracycline have been appearing continuously since the 1980s. MRSA disseminated nosocomially in hospitals today are more than 90% resistant to fluoroquinolones, so that their therapeutic use eliminates nonresistant Staphylococcus aureus and thus represents a risk factor for extensive colonization with MRSA. Of the tested isolates, 0.05% were resistant to quinupristin/dalfopristin. Whereas all MRSA tested by NRZ [National Reference Center for the Surveillance of Nosocomial Infections] for staphylococci in Germany were susceptible to linezolid, the development of resistance is conceivable for this active substance as well. MRSA with a reduced susceptibility to glycopeptides (GISA) are still rare in Germany, as is resistance to the possible combination partners of glycopeptides (rifampicin about 2%, fusidic acid sodium about 2.5%). However, under conditions of continuing selection pressure by glycopeptide antibiotics, it is to be expected that MRSA with resistance to glycopeptides (GISA phenotype) as well will spread increasingly.

Other multiresistant bacterial strains, involved in hospital infections, are vancomycin-intermediate susceptible Staphylococcus aureus (VISA) strains, vancomycin-resistant Staphylococcus aureus (VRSA) strains, vancomycin/glycopeptide-resistant enterococci (VRE, GRE), penicillin-resistant pneumococci, and multiply resistant gram-negative bacteria.

Apart from combating infections with such bacteria by antibiotics, it has become evident in the last decade that for infection prevention it is possible and sensible to eliminate the undesirable bacterial flora by means of antiseptic preparations in order to prevent an infectious disease. Such sanitizing of healthy bacterial carriers is reflected in the terms, decontamination or decolonization. Various antiseptic active substance such as biguanides, bispyridines, chlorhexidine, benzalkonium chloride, or quaternary ammonium compounds are used for this purpose in practice.

Bacteriophages are known as potential alternatives both to antibiotic MRSA therapy and for the antiseptic decontamination of MRSA carriers. Their lytic phenomenon was described for the first time in 1915 by Twort and independently in 1917 by d'Herelle. Bacteriophages were identified as viruses by advances in molecular genetics. They infect specifically only one bacterial species in each case by injecting their DNA into the bacterial cell. They then shift the bacterial metabolism completely to the intracellular de novo synthesis of up to 200 new phages and release this next generation during the breaking up of the bacterial cell. In about 1 part per thousand of bacterial cells, the phage DNA is merely integrated into the bacterial genome (temperate phages), passed on during bacterial division, and becomes virulent again only under special environmental conditions.

Phage therapy was used for the first time against Staphylococcus aureus in 1921 [Bruynoghe R & Maisin J, La Presse Médicale 1921, 1195-1193], whereby infected surgical wounds healed within 48 hours. In the following world war years, bacteriophages were used in postoperative wound infections because of the lack of antibiotics or because of sulfonamide resistance. Spontaneous bacterial resistance during phage therapy was reported for the first time in 1943 (Luria S E & Delbrück M, Genetics 1943, 28, 491-511). There are only about 30 publications on phage therapy, particularly from Eastern Europe, from the Golden Age of antibiotic therapy between 1966 and 1996; phages were used in emergency resistance situations and in the treatment and prophylaxis of postoperative wound infections. More recent studies in Staphylococcus aureus-infected mice corroborated repeatedly in animal experiments the effectiveness of phage therapy (Matsuzaki et al., Journal of Infectious Diseases 2003, 187, 613-624).

Problems during the use of phage therapy as an antibiotic substitute result primarily from the low stability of bacteriophages in the body, because they are eliminated within a short time by macrophages as foreign bodies.

At present, two basically different approaches to phage therapy are pursued: On the one hand, efforts are made to create more effective phage types, omnipotently effective if possible, by means of genetic modifications (Merril C R et al., Nature Reviews Drug Discovery 2003, 2, 489-497; Krylov V N, Russian Journal of Genetics 2001, 37, 715-730; Broxmeyer L et al., Journal of Infectious Diseases 2002, 186, 1155-1160). In contrast to this is the “classical treatment approach” with naturally occurring phages, isolated from the environment, which are effective as mixtures against various bacterial isolates of a species or against different bacterial species (Barrow P A & Soothill J S, Trends in Mircobiology 1997, 5, 268-271; Soothill J S, Journal of Medical Microbiology 1992, 37, 258-261).

In U.S. Pat. Appl. No. 2002/0001590 A1, selected bacteriophages are described from the family Myoviridae, specifically the species of Twort, which are capable of effectively inhibiting or killing MRSA strains. The phages are used in an aqueous solution or a buffer or in a polymer matrix.

Another study [Vautz D. et al.: Lytic effectiveness of MRSA specific phages against the 41 defined national reference strains and 143 clinical MRSA isolates. ZfW (2007) 5:280-287] was based on the question on the qualitative extent to which there are specifically active phages against the group of all 41 MRSA in the German National Reference Center of the Robert Koch Institute in Wernigerode. The phages were exclusively mixtures of natural, i.e., genetically unmodified, viruses. In this regard, phage solutions were found that were active specifically against all 41 MRSA strains.

The problem of nosocomial infections relates to clinical personnel as well who, without being ill themselves, are carriers and therefore disseminators of bacteria. Further, this problem also relates to medical devices, furniture, and fixtures, which may be colonized by bacteria including multiresistant strains. This can also affect the entire hospital building.

The primary goal of the invention, therefore, is to provide a bacteriophage preparation, which is highly effective against at least one bacterial strain, preferably against all known multiresistant bacterial strains, has a good shelf life, and can be distributed well over the surfaces to be treated, including poorly accessible places.

This goal is achieved by a bacteriophage preparation according to claim 1. Advantageous embodiments and refinements of said bacteriophage preparation are described in the dependent claims.

Another goal of the invention is to provide a bacteriophage preparation for producing a medication for therapeutic or preventive antibacterial use, which can be employed in particular in wounds and wound areas, in the nose-throat area, in the skin and mucous membrane area, urogenital area, and in the eye area.

Another goal of the invention is to provide a disinfectant suitable for antibacterial applications, which is suitable, for example, for disinfecting the hands of physicians and clinical personnel, but also for disinfecting objects.

These goals are achieved by the features indicated in claims 12 through 20.

The function of the surface-active substances, on the one hand, is to enable or accelerate the diffusion or penetration of bacteriophages to the bacteria on the skin, in wounds, or other likewise inaccessible places and cavities in the body (e.g., nose-throat or urogenital area) and on inaccessible surfaces, such as, e.g., rough surfaces or narrow crevices. A drastic reduction in bacteria, more effective compared with the prior art, is achieved in this way.

A cationic surfactant, such as polyhexanide, has proven especially good as the surface-active agent.

A good shelf life and an increase in effectiveness is achieved by the alkaline buffer solution indicated in claim 1, whereby it has turned out that bacteriophages can not only be stored better in an alkaline environment in comparison with a neutral or acidic pH (i.e., with lower losses of activity), but also have a much higher effectiveness against the bacteria to be lysed in the alkaline range with a pH above 7.5, e.g., in combination with polyhexanide.

Very low polyhexanide concentrations have only an inhibiting effect on bacteria but do not kill them. On the other hand, bacteriophages are not degraded at these polyhexanide concentrations and their activity is also not hampered, so that the bacteria inhibited by polyhexanide can continue to be lysed by the bacteriophages without this being impaired.

In practical applications, the bacteria are therefore quickly killed either by the locally high concentrations of polyhexanide or inhibited by polyhexanide at very low concentrations and then lysed by the bacteriophages.

The successful bactericidal effect of the bacteriophages depends on whether they can find a suitable receptor on the surface of the bacteria. In this regard, the combination of bacteriophages with surfactants, enzymes, alcohols, cleansing substances such as alginates, and low-frequency ultrasound has proven to be positive. These combination partners improve the possibility of penetration of both antiseptic inhibitors and bacteriophages to the bacteria or, in the case of low-frequency ultrasound, the reaching of their specific receptors by the mechanical/mechano-acoustic action.

Because antimicrobial substances in very low concentrations achieve an inhibiting but not destructive effect, the bacteriophages are enabled to utilize the inhibited bacterial cells for replication and to lyse these in the end.

Further, the combination of bacteriophages with antimicrobially acting chemical substances even in a much lower concentration range (of the inhibiting effect) compared with the prior art has the effect that known gaps in the effectiveness of antimicrobial chemical substances are closed by the bacteriophages.

Further, it can be determined that a substantial improvement in action occurs during the disinfection when the bacteriophage preparation is applied with low-frequency ultrasound, which is preferably within the frequency range below 120 kHz and has an output power in the range of 0.05 to 1.5 W/cm². A better distribution of the bacteriophage preparation over the surface to be disinfected and loosening of dirt particles are achieved in this way, so that the bacteriophages have better access to the bacteria to be combated.

A further improvement of the effect is obtained in that the bacteriophage preparation is heated before or during the application to a higher temperature, which is preferably within the range between 20° C. and 45° C.

Therefore, colonizations or infections by bacteria, particularly colonization of the skin, mucous membranes, body openings and cavities, or wounds, as well as other inaccessible places on the body, e.g., by antibiotic-resistant bacteria such as particularly methicillin-resistant Staphylococcus aureus (MRSA), are drastically reduced with the present invention.

A cationic surfactant is used preferably as the surface-active substance. Other substances with an antiseptic effect, cleansing substances, alcohols, and/or enzymes can also be used.

A “bacteriophage pool” is used preferably, which in this regard has at least one species-specific bacteriophage population with at least one polyvalent bacteriophage strain, which acts specifically against the colonizing bacteria.

Mixtures of bacteriophages, which are effective against different bacterial species in the particular case, can be used as a bacteriophage pool against colonization by different bacterial species.

The bacteriophage populations consist preferably of genetically unmodified bacteriophages. Naturally occurring bacteriophages are greatly preferred. On the other hand, genetically modified bacteriophages naturally may also be used.

Bacteriophage populations, which are then active against different bacterial species, can also be combined in the “bacteriophage pool.”

Lytically active bacteriophages, lysogenic bacteriophages that enter the lytic cycle at a later time, and non-lytic bacteriophages that produce substances harmful to bacteria are regarded as suitable for the “bacteriophage pool.” Lytically active bacteriophages are greatly preferred.

Bacteriophages can also be recovered from hospital wastewater in a manner known to the person skilled in the art and tested for activity against at least one bacterial reference strain, preferably against 1 each of 5 to 20 bacterial reference strains with the drop test and/or the plaque-forming-unit [PFU] test for effectiveness.

Polyvalent bacteriophages, which are effective against different isolates of a species, are preferred.

The preferably lytically active bacteriophages are cultures in suitable bacteria, preferably the species Staphylococcus and Pseudomonas. The resulting lysates are treated further according to known methods to produce a bacteriophage pool therefrom. Said pool should no longer contain any living organisms, toxins, or bacterial cell wall fragments. For example, the lysates can be purified by known methods such as ultrafiltration and/or ultracentrifugation to meet these requirements.

The bacteriophages or other dosage forms of the invention can be packaged in ampoules or other containers. The approximate bacteriophage titers in the container can be determined by determining the predilution suitable for lysing a certain number of bacteria of a test strain. In an embodiment of the invention, bacteriophage pools are prepared with at least 10³ PFU, preferably 10⁶ to 10¹⁰ PFU, highly preferably 10⁷ to 10⁹ PFU of specifically active bacteriophages.

To determine the species specificity of the bacteriophage pools, the bacteriophages are tested in the lysis test with suitable reference strains of the various bacterial species.

The bacteriophage pool is stabilized by combinations of buffering salts in the alkaline pH range, pH values between 7.5 and 9.5 being possible, but pH 8 to pH 9.5 being preferred, and pH 8.2 to pH 9 greatly preferred.

The bacteriophage pool is used in conjunction with at least one surface-active substance. Suitable surface-active substances comprise, inter alia, surfactants, alcohols, antiseptic and disinfecting active substances, cleansing active substances, enzymes, preservatives, low-frequency ultrasound application, and/or combinations thereof.

Suitable surfactants are preferably amphoteric surfactants such as sultaines or betaines. In this regard, it is preferred to use betaines with alkyl chains of 5 to 21 C atoms, preferably 10 to 15 C atoms, and further preferably 11 to 13 C atoms. Undecylenic acid amidopropyl betaine and cocamidopropyl betaine are highly preferred. The surfactants are used in this regard for combination with bacteriophages in a concentration of 0.001 to 10.0% by weight, preferably 0.003 to 5.0% by weight, further preferably 0.005 to 2.0% by weight, and highly preferably 0.005 to 0.1% by weight.

Suitable alcohols are water-soluble alcohols, preferably C1 to C4 alcohols. Ethanol, isopropanol, and n-propanol are highly preferred. The alcohols in this regard for combination with bacteriophages are added in a concentration of 0.1 to 25.0% by weight, highly preferably 1.0 to 10% by weight.

The suitable antiseptic, disinfection agents, and preservatives are preferably especially tissue-compatible substances; especially preferred are biguanides, bispyridines, phenoxyethanol, quaternary ammonium compounds, tosylchloramide sodium, and combinations thereof. Especially preferred of the biguanides are polyhexamethylene biguanide (polyhexanide) and chlorhexidine. Especially preferred of the bispyridines is octenidine dihydrochloride. Especially preferred of the quaternary ammonium compounds is benzalkonium chloride.

The antiseptic disinfectants and preservatives are added during phage preparation in a concentration of 0.000001 to 1.0% by weight. In this regard, the biguanides are used preferably in a concentration of 0.000001 to 1.0% by weight, further preferably 0.000001 to 0.3% by weight. The bispyridines are used preferably in a concentration of 0.01 to 5.0% by weight, further preferably 0.5 to 2.0% by weight. Phenoxyethanol is used preferably in a concentration of 0.05 to 5.0% by weight, further preferably 0.5 to 2.0% by weight. The quaternary ammonium compounds are used preferably in a concentration of 0.01 to 10% by weight, further preferably 0.05 to 1.0% by weight. Tosylchloramide sodium (European Pharmacopoeia 4, “Chloramine T”) is used further preferably as 0.001 to 1% by weight, preferably 0.01 to 0.5% by weight.

Enzymes and alginates are used as cleansing agents individually or together with the bacteriophage pool especially in the treatment of wounds.

Preferred enzymes are trypsin, collagenases NB 1 and NB 4, pancreatin, as well as streptase, urokinase, and lipoxidase. The enzymes are added to the bacteriophage pool in the following concentration:
lipoxidase, 0.01 to 1.0 mg/mL, preferably 1.0 mg/mL
trypsin, 0.2 to 4 mg/mL, preferably 2 mg/mL
streptase, 5,000 to 100,000 IU/mL, preferably 10,000 IU/mL
urokinase, 5,000 IU/mL
NB 1 collagenase, 1 to 10 PZ-U/mL, preferably 6 PZ-U/mL
NB 4 collagenase, 1 to 10 PZ-U/mL, preferably 6 PZ-U/mL
pancreatin, 0.5 to 5 mg/mL preferably 2.5 mg/mL

The following alginates can be added to the bacteriophage pool:
sodium alginates
sodium-calcium alginates
calcium alginates

The described formulations can be prepared by known methods for preparing formulations, which are used for application to surfaces as medical products, preferably to the skin and wounds.

Low-frequency ultrasound in the frequency range <120 kHz, preferably 30-100 kHz, with output powers between 0.05-1.5 W/cm² exhibited additive and, in part, synergistic effects in the combination with bacteriophages and/or polyhexanide during application to the skin, mucous membranes, and wounds.

The application of therapeutic ultrasound of 0.8-3 MHz in subaqual use to stimulate wound healing has been described many times in the literature [Radandt, R. R.: Low-frequency ultrasound in wound healing. Phys Med Rehab Kuror. Thieme Verlag Stuttgart/New York, ISSN 0940-6689 (2001) 11:41-50]. It is known from this that ultrasound influences, e.g., the wound healing process on three levels—that of the mechanical effect on the tissue surface, a mechano-acoustic effect on the microorganisms settling there, and thermal and non-thermal effects in deeper tissue layers.

In particular, the effect of ultrasound between 70 kHz and 10 MHz on the killing of bacteria P. aeruginosa in biofilms in combination with an antibiotic (gentamicin) has been studied in vitro [Qian Z, Sagers R D, Pitt W G. The effect of ultrasonic frequency upon enhanced killing of P. aeruginosa biofilms. Ann Biomed Eng 1997; 25, 1:69-76]. In this regard, the result was that the ultrasound treatment alone did not cause a significant reduction in microorganisms. With the combination of ultrasound and the antibiotic, however, a significant increase in activity compared with the control group (antibiotic alone) was demonstrated at all frequencies: There was a clear correlation to the ultrasound frequency, an increase in effect >250% being found below 70 kHz (“bioacoustic effect”).

The following applications of low-frequency ultrasound can be combined with the bacteriophage pool:
frequency range <120 kHz with output powers of 0.05-1.5 W/cm²
frequency range <100 kHz with output powers of 0.05-1.5 W/cm²
frequency range <70 kHz with output powers of 0.05-1.0 W/cm²

Suitable application forms for the bacteriophage pool in conjunction with surface-active substances or low-frequency ultrasound in this regard are solutions, irrigation solutions, (wound) dressings made of textile materials or synthetics, as well as gels, or combinations thereof. Sterile application forms are preferred. In this case, sterilization occurs by conventional methods known to the skilled person.

The suitable solutions are preferably aqueous solutions and may contain other active substances, particularly wash-active and antiseptic substances. Preferably, these solutions can be used specifically preventively for antiseptic disinfection of surfaces (e.g., in operating rooms or intensive care units of hospital).

The suitable irrigation solutions are preferably aqueous solutions, which may contain additional active substances preferably suitable for disinfection and well tolerated by the skin. Moreover, the irrigation solutions may contain buffered iso-osmotic solutions with a suitable pH and a physiological salt content.

The suitable (wound) dressings can consist of textile materials, e.g., conventional cotton gauze, or synthetic fiber materials or other bulk agents.

Suitable gels contain preferably glycerol or polymers or a combination thereof. The polymers are preferably derivatives of cellulose acetate; preferred further are hydroxyalkylcellulose acetate, particularly hydroxyethylcellulose acetate and hydroxypropylcellulose acetate, as well as polymers of acrylic acid, and preferred further are acrylic acid homopolymers, which may be crosslinked with pentaerythritol allyl ether, sucrose allyl ether, or propylene allyl ether (carbomer).

Suitable gels contain glycerol in a concentration of 1.0 to 10.0% by weight, preferably 5.0 to 8.0% by weight, and/or polymers in a concentration of 0.1 to 10.0% by weight, preferably 2.0 to 6.0% by weight.

The bacteriophage preparations of the invention in combination with surface-active substances are used for producing a medication for the therapeutic or preventive antibacterial application to the skin and mucous membranes, preferably in the nose-throat area and in the urogenital area, in wound areas, and in the eye area.

The formulations and uses of the invention are suitable for eliminating bacteria, such as, for example, strains of Staphylococcus, Streptococcus, Enterococcus, Pseudomonas, Enterobacter, coliform bacteria, Klebsiella, Proteus, Listeria, or Salmonella. The formulations and methods are especially suitable for eliminating antibiotic-resistant bacterial strains preferably of Staphylococcus and Pseudomonas, especially preferably of Staphylococcus aureus and of Pseudomonas aeruginosa. The formulations and methods are especially highly suitable for eliminating methicillin-resistant Staphylococcus aureus (MRSA). In a preferred embodiment of the invention, the formulations and methods are used to remove MRSA colonizations of the skin preferably in inaccessible places such as the nose-throat area or in wounds.

The described embodiments of the invention will be described in greater detail by the following example. The present invention is not limited to said example, however.

### EXAMPLE

for producing a preparation of bacteriophages (here: effective against Staphylococcus aureus) and (here: a) surface-active substance:

### 1. Bacteriophage Isolation and Replication

The necessary reference bacteriophages and the reference bacterial strains were obtained from the strain collection.

To recover new bacteriophages, a liquid sample from hospital wastewater can be combined with CaCl₂ and MgCl₂, filtered, and then sterile-filtered. The liquid sample is subjected to a PFU test to detect bacteriophages lytically active specifically against Staphylococcus aureus. For this purpose, the sterile-filtered sample is added in each case to a log-phase culture of methicillin-resistant Staphylococcus aureus diluted with liquid nutrient medium.

For replication of the specific phages against Staphylococcus aureus, a fresh bacterial culture of the particular reference strain in the log phase is added to the bacteriophages in a shaken Erlenmeyer flask. To prevent resistance formation, the clarified suspension is then centrifuged and the supernatant sterile-filtered. The replication step is repeated until a concentration of 10¹⁰ phages/mL is achieved in the PFU test.

This procedure is repeated with all Staphylococcus aureus strains and their specific phages until all relevant bacteriophages (bacteriophage pool) are detectable in the mixture in the concentration of 10¹⁰/mL in the PFU test.

### 2. Determination of Action Specificity

To determine the species specificity, the obtained phage suspensions were used in the PFU test against the bacterial strains E. coli ATCC 11229 and P. aeruginosa ATCC 15442. In this regard, no lytic activity of the employed phage suspension against the control strains may be detected.

### 3. Production of the Preparation (Phage Preparation)

To produce the phage preparation from the bacteriophage pool and the surface-active substance, solutions from the pooled phage suspension with an end concentration of 10⁷ bacteriophages/mL and 0.1% by weight undecylenic acid amidopropyl betaine are stabilized with an alkaline buffer, incubated, and sterile-filtered.

### 4. Concentration of Bacteriophages in the Preparation

The phage preparation is tested for its activity in regard to all relevant Staphylococcus aureus reference strains in the PFU test. In this regard, the full effectiveness of the pooled phage suspension against all relevant reference strains could be determined.

### 5. Adverse Reaction Tests

The produced batch is also tested for toxic and irritant effects, as well as with respect to enterotoxins (a-g, h, and i), exfoliative toxins a and b, the toxin of the toxic shock syndrome, and endotoxins.

1. A bacteriophage preparation for the treatment of colonizations caused by bacteria (for example, contaminations, invasions, infections, and infectious diseases), characterized in that the preparation has at least one bacteriophage specifically effective against at least one bacterial strain in an alkaline buffer solution in combination with at least one surface-active agent;
wherein the bacteriophage is lytically active against at least one bacterium selected from among Staphylococcus species, Pseudomonas species, Streptococcus species, Enterococcus species, Enterobacter species, Klebsiella species, Proteus species, Listeria species, and Salmonella species; and
wherein the surface active agent is a cationic surfactant.

2. (canceled)

3. (canceled)

4. The bacteriophage preparation according to claim 1, characterized in that the surface-active agent is polyhexamethylene biguanide (polyhexanide).

5. The bacteriophage preparation according to claim 4, characterized in that the pH of the bacteriophage preparation is stabilized by at least one buffering salt to an alkaline range between pH 7.5 and pH 9.0.

6. The bacteriophage preparation according to claim 1, characterized in that at least one surface-active agent is present in the preparation in a concentration of 0.000001 to 20.0% by weight, based on the entire bacteriophage preparation.

7. The bacteriophage preparation according to claim 1, characterized in that the preparation is present as a solution or suspension.

8. The bacteriophage preparation according to claim 1, characterized in that the preparation is present in gel form.

9. The bacteriophage preparation according to claim 8, characterized in that the preparation comprises glycerol or a polymer selected from the group consisting of derivatives of cellulose acetate and polymers of acrylic acid.

10. The bacteriophage preparation according to claim 9, characterized in that glycerol is present in a concentration of 1.0 to 10.0% by weight, based on the entire bacteriophage pool.

11. The bacteriophage preparation according to claim 10, characterized in that the polymer is present in a concentration of 0.1 to 10.0% by weight, based on the entire bacteriophage pool.

12. The bacteriophage preparation according to claim 1, characterized in that the preparation is present in textile fibers or synthetic fibers, or as a bulk material.

13. Use of a bacteriophage preparation according to claim 2 for producing a medication for therapeutic or preventive antibacterial use.

14. The use according to claim 13 for application in wounds and wound areas.

15. The use according to claim 13 for application in the nose-throat area.

16. The use according to claim 13 for application in the skin and mucous membrane area.

17. The use according to claim 13 for application in the urogenital area.

18. The use according to claim 13 for application in the eye area.

19. The use of a bacteriophage preparation according to claim 2 for the production of a disinfectant for antibacterial applications.

20. A disinfection method characterized by the application of the bacteriophage preparation according to claim 2 with low-frequency ultrasound with a frequency range <120 kHz and an output power of 0.05-1.5 W/cm².

21. The disinfection method according to claim 20, characterized in that the bacteriophage preparation is heated before application to a temperature between 22° C. and 45° C.
